(19) United States
(12) Patent Application Publication
(10) Pub. No.: US 2007/0078494 A1
(43) Pub. Date: Apr. 5, 2007

(54) Method and apparatus for controlling motility of gastrointestinal organs for the treatment of obesity

(76) Inventor: Martin P. Mintchev, Calgary (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/355,455

(22) Filed: Feb. 15, 2006

Related U.S. Application Data
(60) Provisional application No. 60/719,806, filed on Sep. 23, 2005.

(51) Int. Cl. A61N 1/00

(52) U.S. Cl. 607/40

(57) ABSTRACT

Techniques are provided for controlling motility of the gastrointestinal tract using an implantable medical system. In one technique, electrical stimulation is applied to a set of electrodes implanted in the gastrointestinal tract to induce a contraction that artificially propagates in a retrograde direction. The invocation of retrograde contractions or reverse peristalsis provides at least partial control over gastric emptying and thus, indirectly, satiety and food intake.

### CROSS REFERENCE TO RELATED APPLICATIONS

This application claims the benefit under 35 USC 119(e) of Provisional patent application No. 60/719,806 filed Sep. 23, 2005.

### FIELD OF THE INVENTION

The invention generally relates to implantable medical devices and more particularly relates to a method and apparatus for controlling the motility in the organs of the gastrointestinal tract.

### BACKGROUND OF THE INVENTION

Control of gastrointestinal motility is of interest to medical practitioners involved in the treatment of disorders of the gastrointestinal tract and in the treatment of conditions related to the function of the gastrointestinal tract such as obesity. The gastrointestinal tract generates coordinated contractions that are necessary for the emptying of the stomach. Gastric emptying plays an important role in regulating food intake. For example, contraction-mediated gastric distention acts as a satiety signal to inhibit food intake and rapid gastric emptying is closely related to overeating and obesity.

Gastric motility (contractile activity) is in turn regulated by myoelectrical activity of the stomach. Gastric myoelectrical activity includes two components, slow waves and spike potentials. Typically, slow waves are always present and occur at regular intervals whether or not the stomach contracts. Slow waves originate in the proximal stomach and propagate distally toward the pylorus. A strong contraction occurs when a spike potential (similar to an action potential), is superimposed on the gastric slow wave.

A variety of gastrointestinal electrical stimulation systems for the treatment of gastrointestinal motility disorders have been proposed. The systems typically attempt to control gastrointestinal motility by enhancing or manipulating the spontaneously existing myoelectric gastrointestinal activity to entrain or stimulate gastrointestinal motility.

### SUMMARY OF THE INVENTION

In accordance with aspect of the present invention a method for controlling motility in a gastrointestinal tract includes applying gastric electrical stimulation to a set of electrodes implanted in a pre-pyloric region of the gastrointestinal tract to induce a circumferential contraction that propagates in a retrograde direction.

In accordance with another aspect of the present invention a method for controlling motility in a gastrointestinal tract includes applying gastric electrical stimulation to a set of electrodes implanted in a pre-pyloric region of the gastrointestinal tract to induce paresis in at least a portion of the gastrointestinal tract.

In accordance with a further aspect of the invention, there is provided a set of electrodes suitable for implantation a predetermined region of a gastrointestinal tract; and a controller adapted to deliver electrical stimulation to the set of electrodes to induce a circumferential contraction that propagates in a predetermined direction.

### BRIEF DESCRIPTION OF THE DRAWINGS

The above and further features, advantages and benefits will be apparent upon consideration of the present description taken in conjunction with the accompanying drawings, in which:

FIG. 1 is a simplified diagram of one embodiment of an implantable stimulation device in electrical communication with a patient's gastrointestinal tract in accordance with one embodiment of the present invention;

FIG. 2 is a timing diagram illustrating parameters of a train of neural electric gastric stimulation pulses in accordance with one embodiment of the present invention;

FIG. 3 is a simplified block diagram of a multi-channel implantable stimulation device configured to provide stimulation to the gastrointestinal tract in accordance with one embodiment of the present invention;

FIG. 4 is a simplified diagram of one embodiment of a rechargeable implantable stimulation system in electrical communication with a patient's gastrointestinal tract in accordance with one embodiment of the present invention;

FIG. 5 is a simplified block diagram of a multi-channel, rechargeable implantable stimulation device suitable for use in the system of FIG. 4 in accordance with one embodiment of the present invention;

FIG. 6 is a simplified block diagram of an external controller suitable for use in the system of FIG. 4 in accordance with one embodiment of the present invention;

FIGS. 7(a-d) graphically illustrate electrode configurations suitable for use with the implantable stimulation device of FIGS. 1 and 4 in accordance with one embodiment of the present invention;

FIG. 8 graphically illustrates an artificial retrograde contraction induced by a single electrode set as measured at three distinct locations on the GI tract in accordance with one embodiment of the present invention;

FIGS. 9(a-b) graphically illustrates the strength of an artificial retrograde contraction induced by a single electrode set as measured at two distinct locations on the GI tract as a function of the duration of stimulation in accordance with one embodiment of the present invention;

FIGS. 10(a-b) graphically illustrates the strength of an artificial retrograde contraction induced by a single electrode set as measured at two distinct locations on the GI tract under sustained stimulation in accordance with one embodiment of the present invention;

FIG. 11 graphically illustrates the force as a function of time of underlying intrinsic contraction measured at three distinct locations on the GI tract;

FIG. 12 graphically illustrates the force measured at three locations on the GI tract as a function of time resulting from the induction of paresis into that portion of the GI tract through NGES stimulation delivered through a single electrode set in the pre-pyloric areas in accordance with one embodiment of the present invention;

FIG. 13 graphically illustrates an additional electrode configuration suitable for use with the implantable stimulation device of FIGS. 1 and 4 in accordance with one embodiment of the present invention; and

FIG. 14 graphically illustrates an overlapping stimulation pattern for dual electrode sets in accordance with one embodiment of the present invention.

### DETAILED DESCRIPTION OF PREFERRED EMBODIMENTS OF THE INVENTION

The invention is described below, with reference to detailed illustrative embodiments. It will be apparent that the invention may be embodied in a wide variety of forms, some of which may be quite different from those of the disclosed embodiments. Consequently, the specific structural and functional details disclosed herein are merely representative and do not limit the scope of the invention.

An implantable stimulation device, in accordance with one embodiment of the present invention provides neural gastric electrical stimulation (NGES) to produce contractions in the vicinity of the implanted electrodes. The present invention therefore provides full and direct control over gastric contractions, while overriding any spontaneously existing electromechanical events.

The advantages of the present invention may be best understood in connection with an illustrative stimulation device that is capable of being used in connection with the various embodiments that are described below. It is to be appreciated and understood, however that other stimulation devices, including those that are not necessarily implantable, can be used and that the description below is given, in its specific context, to assist the reader in understanding, with more clarity, the inventive embodiments described herein. In the description that follows, like numerals or reference designators will be used to refer to like parts or elements throughout.

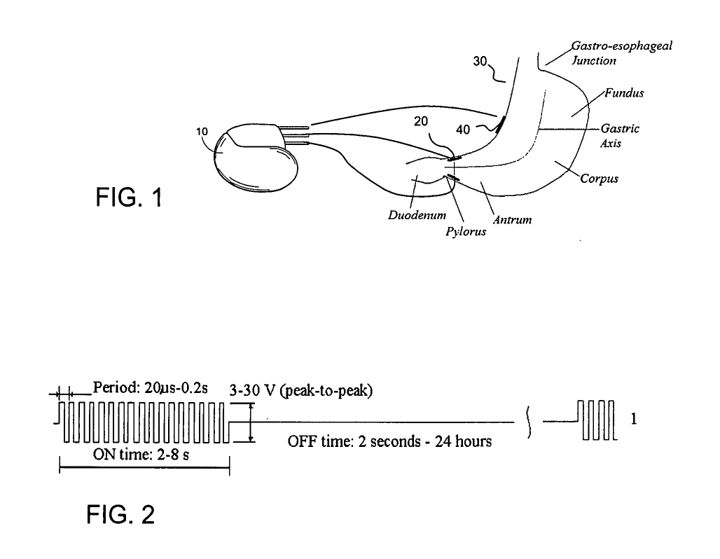

Referring to FIG. 1, in one embodiment of present invention an implantable pulse generator 10 drives one or more sets of electrodes 20 to apply NGES stimulation to a portion of a patient's stomach 30. The implantable pulse generator may also be coupled to one or more sensing electrodes 40 or mechanical sensors that provide feedback regarding the intrinsic myoelectric activity of the stomach as well as the intrinsic and induced contractile activity of the stomach.

Typically, the implantable pulse generator 10 applies NGES stimulation to a portion of the patient's GI tract (e.g. the stomach as illustrated in FIG. 1) to artificially induce a contraction in the area of the implanted electrodes. The external signals supplied to the electrode sets, although synchronized between themselves, are independent from and typically asynchronous with the spontaneously existing myoelectric activity (e.g. the slow waves) in the area of the implanted electrodes. In this embodiment the stimulation signals override the existing myoelectric activity to independently control contractile activity rather than stimulating or enhancing the intrinsic gastric activity.

Referring to the timing diagram of FIG. 2, in some embodiments, the frequency of the stimulation signals ranges from 5 to 50,000 Hz, and the amplitude of the stimulation signals ranges from 3 V peak-to-peak to 30 V peak-to-peak, or from 3 mA peak-to-peak to 60 mA peak-to-peak. The stimulation signals are typically delivered in bursts of pulses with a range of duty cycles.

Further, the train of stimulation pulses may have a variable on time ranging from about 2 to 16 seconds and a variable off time between pulse trains or bursts ranging from 2 seconds to 24 hours in a single session. The voltages, on time and duty cycle used in a particular case will be selected for the particular application and may vary from individual to individual, but will generally supply an amount of energy to the tissue above a clinically effective threshold that can be determined for the individual concerned.

The delivered current requirement for the implantable pulse generator can be estimated with regard to the average total current consumption per unit muscular thickness of GI tissue per electrode pair, which is typically about 3 mA/mm. Typically the thickness of the muscle in the GI tract is in the range of 2.5 mm to 3.55 mm so that the average total current drawn by the tissue will be in the range of 7.5 mA to 10.5 mA.

Referring back to FIG. 1, in some embodiments one or more dedicated sensing electrodes 40 are coupled to the patient's stomach to monitor the underlying myolectrical activity of the stomach. In some embodiment one or more mechanical sensors (e.g., accelerometers, force transducers, strain gauges, or pressure gauges) may be used instead of or in conjunction with the sensing electrodes. The mechanical sensors and sensing electrodes convey feedback signals to a controller in the implantable pulse generator regarding various aspects of the patient's metabolic system, i.e. the detection of the onset of a patient eating, the level of GI activity, as well the approximate volume of food in the stomach.

In some embodiments, the implantable pulse generator utilizes the feedback signals to control the delivery of stimulation signals. The feedback signals include readings from sensors of mechanical and/or electrical activity, which are used to determine the motility of the GI tract. Once the motility of the GI tract is known, an algorithm controls the stimulation signals to maintain the paretic status, or to allow restoration of normal peristalsis in accordance with a dietary schedule. The schedule may restrict the patient's ability to eat to predetermined periods of time throughout the day to control the amount of food a patient ingest between regularly schedule meals. Alternatively or additionally, the implantable pulse generator delivers stimulation signals during one or more meals during the day, so as to reduce the patient's appetite during those meals. In some embodiment, stimulation pulses are then withheld during the remainder of the day, so as to prevent counterproductive remodeling of the stomach.

Further, the implantable pulse generator may utilize the feedback signals to adjust any of the stimulation parameters during a particular meal or between meals throughout the day or throughout the course of a particular treatment. For example, the implantable pulse generator in one embodiment delivers, by way of example, low amplitude pulses during the onset of the ingestion of food and increases the intensity of the delivered stimulation in accordance with the volume of food ingested. Alternatively, the implantable pulse generator may deliver relatively low intensity stimulation during the ingestion of food to mitigate negative side effects such as, for example, nausea that may occur under higher intensity stimulation. In this embodiment the implantable pulse generator may then deliver higher intensity stimulation between meals when the volume of food in the patient's stomach is lower and the patient is less prone to negative side effects.

Although this invention contemplates various feedback algorithms, an exemplary algorithm for determining the restoration of normal peristalsis may involve establishment of motility index threshold levels for each of several implanted force transducers, establishment of amplitude threshold levels for the recognition of peaks of Electrical Control Activity (ECA) and establishment of synchronization patterns between Gastric Electrical Activity (GEA) channels. Exemplary motility threshold levels may be lower, for example 10-20% lower, than the average motility index levels for the force transducers measured postprandially after the post-operative recovery of the patient but before the start of the NGES therapy. Establishment of synchronization patterns should allow for timing tolerances.

In this non-limiting example, the combination of these three conditions can deliver algorithmically a permission to administer another NGES, or conversely, not to do so. For example, if the post-NGES motility index reaches the established motility index threshold, and the GEA channels exhibit synchronized electrical coupling, then normal, spontaneously existing peristalsis has been restored. Therefore, a decision can be made to administer pre-pyloric NGES again, so that the paretic status achieved as a result of the preceding NGES session can be maintained.

In addition, in some embodiments the patient can activate, deactivate, and modify the level of signal application in accordance with a physician's instructions, aspects of the patient's diet, or other factors. Further, the implantable pulse generator may also use sensor feedback signals, e.g., the level of muscle contraction measured by a mechanical sensor or the maintenance of a patient's blood sugar level within a predetermined range to modify the parameters of the stimulation signal to achieve a desired response.

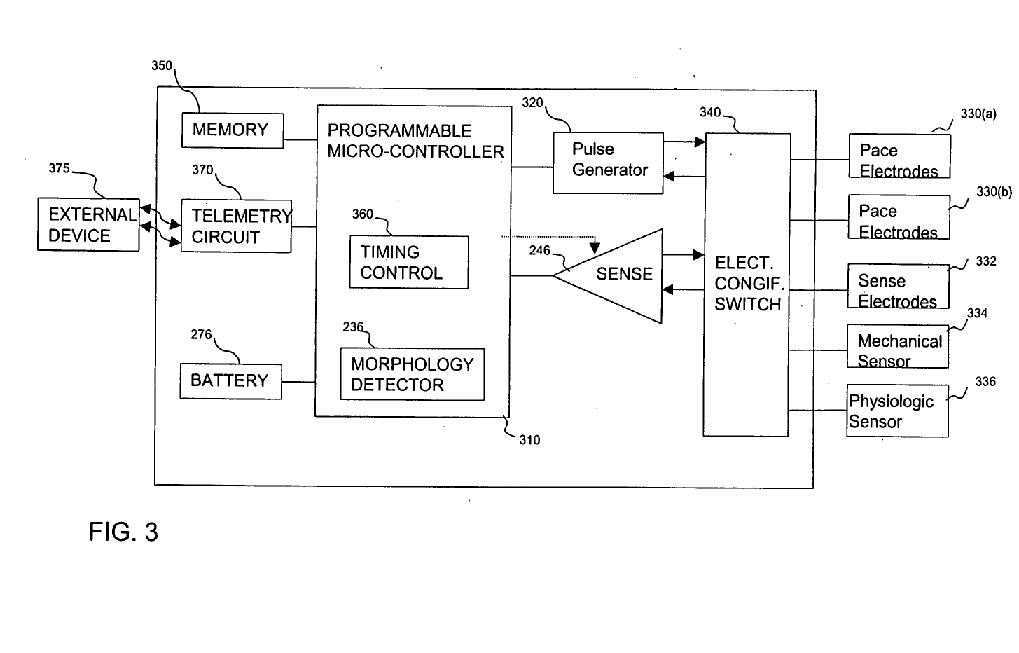

The present invention may be practiced in any of a variety of stimulation devices capable of delivering NGES to the GI tract to produce contractions in the vicinity of the implanted electrodes. For example, FIG. 3 is a simplified block diagram depicting various components of an illustrative stimulation device 10 which is capable of controlling the motility of the GI tract. While a particular multi-channel device is shown, it is to be appreciated and understood that this is done for illustration purposes only. Thus, the techniques and methods described below can be implemented in connection with any suitably configured or configurable stimulation device. Accordingly, one of skill in the art could readily duplicate, eliminate, or disable the appropriate circuitry in any desired combination to provide a device capable of delivering NGES stimulation to produce contractions in the stimulated area.

The stimulation device 10 includes a programmable microcontroller 310 that controls the various modes of stimulation therapy. As is well known in the art, microcontroller 310 typically includes a microprocessor, or equivalent control circuitry, designed specifically for controlling the delivery of stimulation therapy, and may further include RAM or ROM memory 350, logic and timing circuitry 360 and I/O circuitry.

Typically, microcontroller 310 includes the ability to process or monitor input signals (data or information) as controlled by a program code stored in a designated block of memory. The type of microcontroller is not critical to the described implementations. Rather, any suitable microcontroller 310 may be used that carries out the functions described herein. The use of microprocessor-based control circuits for performing timing and data analysis functions are well known in the art.

The stimulation device 10 further includes a pulse generator 320 that generates stimulation pulses for delivery by one or more electrode pairs 330 (a-b) (only two ports shown for clarity) via an electrode configuration switch 340. It is understood that in order to provide stimulation therapy to more than one site on the GI tract, the stimulation device may include independent pulse generators for each stimulation site, multiplexed pulse generators, or shared pulse generators. The pulse generator(s) 320 is controlled by the microcontroller 310 via appropriate control signals to trigger or inhibit the stimulation pulses.

The implantable pulse generator in one embodiment is coupled to one or more sensing electrodes 332, mechanical sensors 334 or physiologic sensors 336 that provide feedback regarding the intrinsic myoelectric activity of the stomach as well as the intrinsic and induced contractile activity of the stomach. The physiologic sensor 336 may also monitor the patient's activity level, posture or other physiologic parameters of the patient to provide further information that may be useful in control of the delivered stimulation pulses.

Microcontroller 310 further includes timing control circuitry 360 to control the timing of the stimulation pulses (e.g., frequency, duty cycle, etc.) which is well known in the art. The microcontroller 310 is also coupled to a memory 350 by a suitable data/address bus wherein the programmable operating parameters used by the microcontroller 310 are stored and modified, as required, in order to customize the operation of the stimulation device 10 to suit the needs of a particular patient. Such operating parameters define, for example, pulse amplitude, pulse duration, electrode polarity, rate, sensitivity, automatic features and wave shape to be delivered to the patient's GI tract within each respective tier of therapy.

Advantageously, the operating parameters of the implantable device 10 may be non-invasively programmed into the memory 350 through a telemetry circuit 370 in telemetric communication via communication link with an external device 375, such as a programmer, trans-telephonic transceiver, or a diagnostic system analyzer. The microcontroller 310 activates the telemetry circuit 370 with a control signal. The telemetry circuit 370 advantageously allows status information relating to the operation of the device 10 (as contained in the microcontroller 310 or memory 360) to be sent to the external device 375 through an established communication link.

The stimulation device additionally includes a power system 380 that provides operating power to all of the circuits shown in FIG. 3. The power supply may take the form of an autonomous battery or an autonomous battery which is rechargeable through a transcutaneous inductive link. In one embodiment the battery may recharged by an external power source, such as, by way of example, an abdominal belt which is periodically worn by the patient (preferably during sleep. Alternatively, the stimulation device may utilize external power source which provides power to the stimulation device via a transcutaneous inductive link during the periods of the desired gastrointestinal organ control.

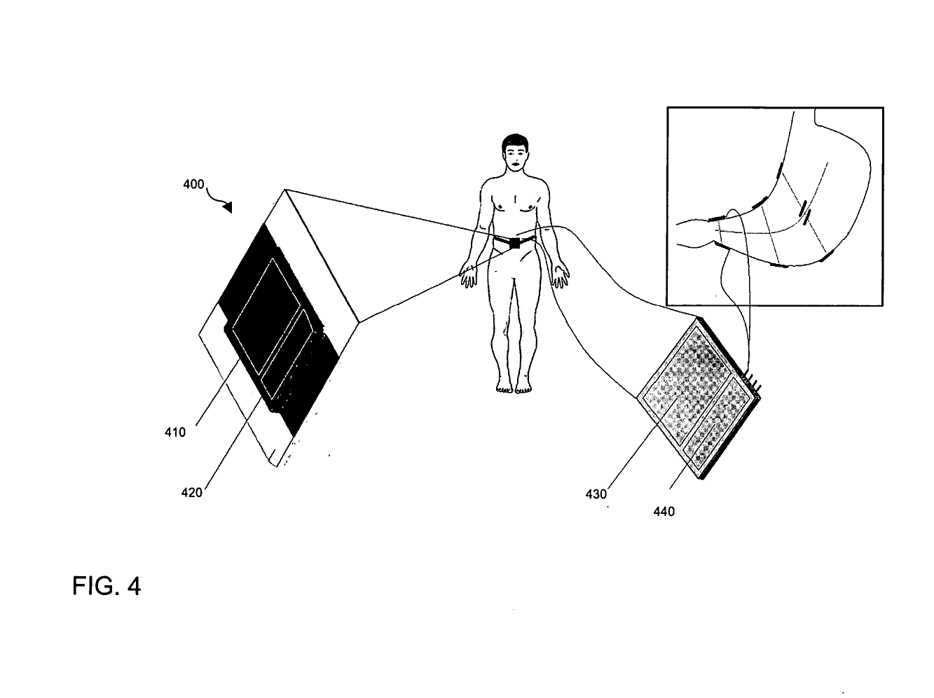

For example, FIG. 4 graphically illustrates a distributed stimulation device with a rechargeable battery for a power source. The external control is administered via an external transmitter 400 (an abdominal belt in the illustrated embodiment), in which the transmitting inductive coil 410 for transcutaneous power transfer is positioned, along with the associated microcontroller-based electronics 420 (see also FIG. 6).

The stimulation device 10 is implanted on the inner side of the abdominal wall and the external power source is coupled to the body in the abdominal area over the center of the implanted stimulation device. In this embodiment the stimulation device includes a receiving coil 430 which is aligned with the transmitting inductive coil 410 of the external device and microcontroller-based electronics 440 (see also FIG. 6). The implanted stimulation device is shown with four channels, and the pyloric channel is coupled to the stomach.

One of skill in the art will appreciate that transmitting and receiving coils is not necessary for an embodiment that utilizes an autonomous non-rechargeable battery-based power supply for the implanted stimulation device. Such an embodiment reduces the inconvenience to the patient by removing the need to wear an external power source to recharge the battery. However, a completely autonomous system may require more frequent explantation of the stimulation device in order to replace the battery.

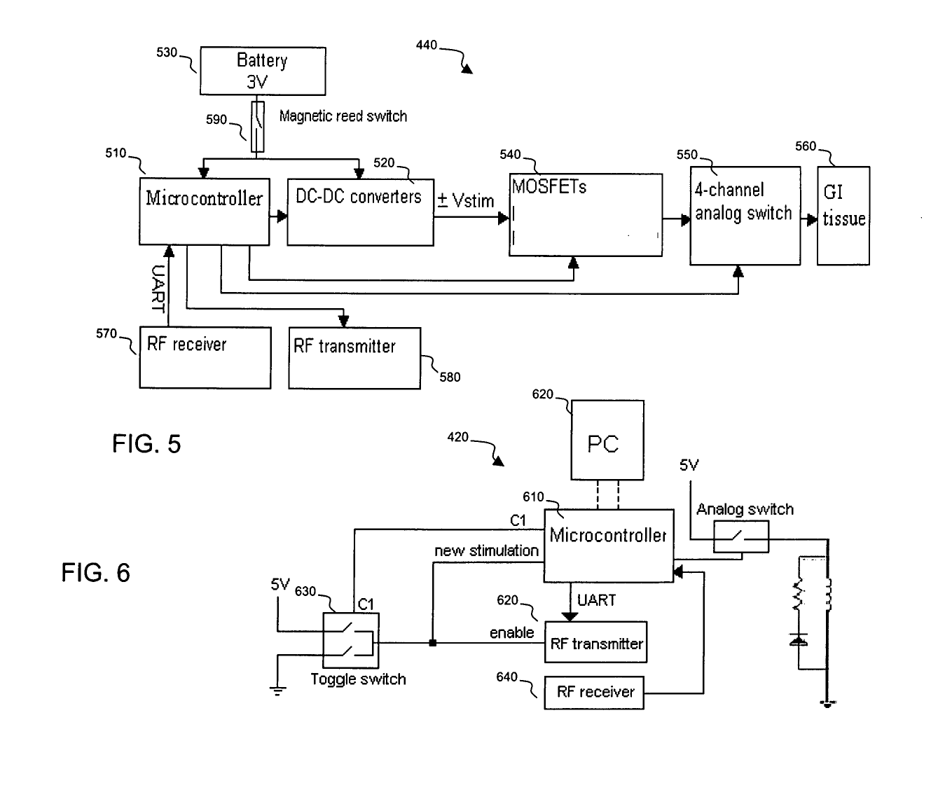

FIG. 5 is a simplified block diagram of an exemplary microcontroller-based electronic circuit 440 suitable for use in an implantable stimulation device with a rechargeable power system. The device of FIG. 5 includes, by way of example, a microcontroller 510 which is programmed to control the various modes of stimulation therapy. For example, in one embodiment the microcontroller 510 controls the generation of digital stimulation pulses through control of the output of a DC-DC conversion stage 520 that converts the voltage supplied by battery 530 (in one embodiment 3V) to the desired stimulating amplitude (Vₛₜᵢₘ).

In one embodiment the microcontroller also generates a digital square wave having an amplitude equal to the battery voltage. In this embodiment a signal conditioner 540 converts the digital square wave produced by the microcontroller into a bipolar analog signal having the same frequency and amplitude as the DC-DC converter output signal, Vₛₜᵢₘ.

In one embodiment the signal conditioner 540 comprises a MOSFET stage having logic transistors which have a low gate threshold voltage that are switched by the digital square wave produced by the microcontroller. These logic transistors then drive the gates of power FETs, which convert the digital square wave to the bipolar analog signal.

In this embodiment the output of the signal conditioner 540 is coupled to a multi-channel analog switch 550 which couples the bipolar analog stimulation signal to one or more sets of stimulation electrodes which are coupled to the GI tissue of the patient's GI tract. The analog switch also isolates each electrode from successive electrode sets.

Microcontroller 510 also determines the duration of each stimulation session and the overlap between successive stimulation channels via the analog switch 550. In some embodiment the microcontroller 510 can vary the stimulation parameters (amplitude, frequency, overlap, and session length) from one stimulation session to another or within a particular stimulation session. The microcontroller 510 is, by way of example, pre-programmed with a set of different values for each stimulation parameter. In addition, the treating physician can specify a default value for each parameter. The treating physician can choose the desired value of each parameter from this pre-determined list of default values using a transcutaneous control link.

In one embodiment the microcontroller utilizes a relatively low clock frequency, such as, by way of example, 20 KHz, to minimize the switching power losses in the microcontroller. Typically, the maximum stimulation frequency will be 500 Hz, resulting in a minimum pulse width of 2 ms. Therefore, a 20 KHz crystal, which has an instruction cycle of 50 μs, is sufficiently large for generating 2ms or slower pulses.

The implantable stimulation device 500 in this embodiment further includes an RF receiver 570 that receives serial wireless data containing the choice of the stimulation parameters from an external portable control unit. This data is transmitted serially and in an asynchronous mode to the microcontroller using a universal asynchronous receiver-transmitter (UART). The data transfer rate (baud rate) is set to 125 bit/s for operation with a crystal frequency of 20 KHz. The microcontroller will sample the data at a suitable rate, such as, by way of example, 16 times the baud rate.

In one embodiment, if the UART does not detect a start bit for data transfer in the first five seconds after power-up, the microcontroller will start a stimulation session using its default parameters. In this embodiment the microcontroller sends a confirmation byte at the onset of stimulation (five seconds after startup) to the external control circuit via an RF transmitter 580. For example, a byte with all one bits is used to represent the onset of stimulation with new parameters, while a byte with all zeros is used to represent the onset of stimulation in accordance with the default parameters.

In one embodiment a portable, microcontroller-based control circuit 410 suitable for use in an external controller is illustrated in the simplified block diagram of FIG. 6. The microcontroller-based circuit 410 allows the user to select the appropriate parameters for delivering NGES to produce contractions in the vicinity of the implanted electrodes.

In one embodiment the external controller 600 is battery-operated and is worn by the patient in an abdominal belt. In this embodiment, a digital wireless transmitter 620 is used to transmit stimulation parameters to an implanted stimulator.

The external controller can also be used to adjust the number of successive stimulation sessions as well as the pause period between the successive sessions. The external controller turns the implanted stimulator on or off for adjustable lengths of time by controlling a normally open magnetic reed switch 590 or the like (see FIG. 5) that is integrated in the implanted device. In some embodiments, the reed switch is in series with the implanted battery to control power delivery to the implanted stimulation device. The external controller turns the magnetic reed switch on by energizing a coil to generate a static magnetic field.

The external controller further includes, by way of example, a switch, such as toggle switch 630, that allows the user to implement either a default stimulation session (using the implanted stimulator's default parameters) or a new stimulation session. In one embodiment the parameters for the new stimulation session are downloaded to the external unit's microcontroller from a PC via an RS232, USB or other suitable link. These parameters are transferred from the microcontroller 610 to the wireless transmitter 620 using the UART line, at a baud rate equal to the data transfer rate of the implanted stimulation device (e.g. 125 bit/s).

The wireless transmitter 620 then transmits this information to the implanted stimulation device. In the case of stimulation with default parameters, the wireless transmitter 620 is disabled and the microcontroller 610 does not send any data to wireless transmitter. In this instance, the microcontroller 610 uses the reed switch to activate the implanted stimulation device. The implanted stimulation device interprets the lack of incoming information from the transcutaneous link as a sign that default stimulation must be performed.

The external controller further includes an RF receiver 640 that receives the ‘confirmation byte’ from the implanted stimulation device. The microcontroller sends a signal to de-energize the coil t+5 seconds after startup, where t represents the time length of each stimulation session.

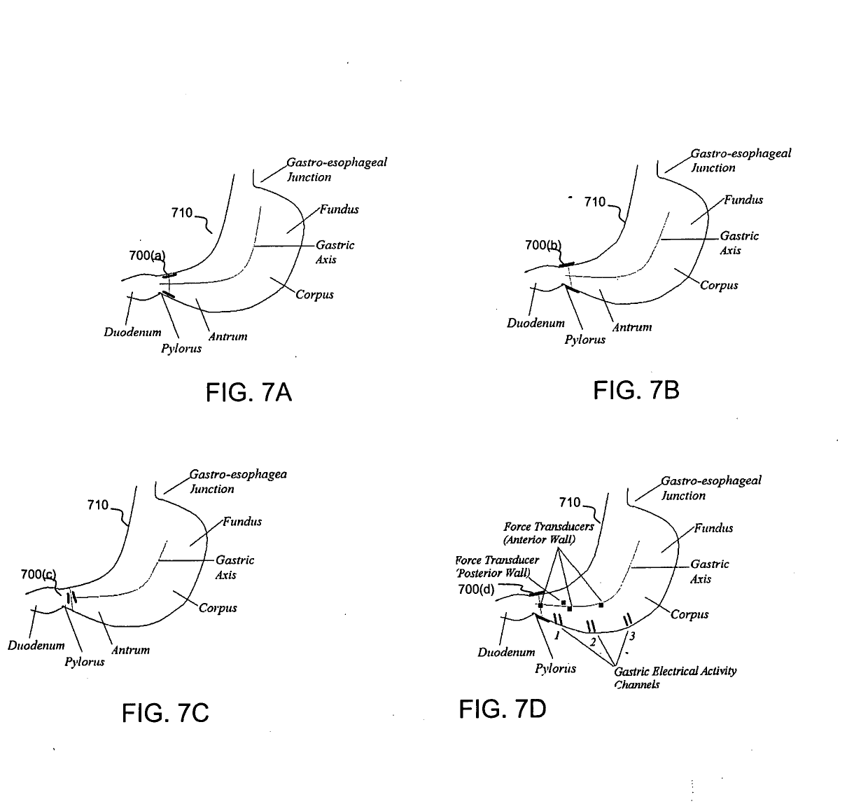

FIGS. 7(a-d) graphically illustrate representative electrode configurations 700(a-d) respectively, for delivering NGES stimulation to portions of the GI tract such as the stomach 710. In some embodiments, the electrodes are implanted in the pre-sphincter area of a GI organ (for example, in the pre-pyloric area of the stomach) either from the serosal or the mucosal side of the organ (e.g. the stomach, the colon, the esophagus, etc.). Typically the electrodes are implanted in pairs, where each pair of electrodes comprises a ground (reference) electrode and the other electrode being the active electrode.

The number of electrode pairs typically varies with the circumference of the organ in the area where the electrodes are implanted. The electrode pairs are typically implanted along an imaginary line perpendicular to the organ axis, so that a circumferential electrode field is established between the pairs of electrodes, when stimulation voltage is applied to them. One or several local electrode sets can be implanted along the axis of the gastrointestinal organ.

The electrodes can be collinear with the longitudinal axis of the GI organ as illustrated in FIGS. 7(a-b) or perpendicular to the longitudinal axis as illustrated in FIG. 7(c). The electrodes are typically implanted on opposing sides of the GI organ, so that the electric current produced as a result of the stimulation flows circumferentially through the GI organ segments on the anterior and the posterior wall located between the implanted electrodes.

The length of the electrodes is, by way of example, between about 0.1 and 10 cm. The distance between electrode sets is, by way of example, between about 1.5 and 10 cm. In some embodiments the electrodes are implanted so as to ensure that the electrodes within a given electrode pair and electrodes from adjacent electrode sets do not contact one another. In some embodiment the minimum distance between electrodes is greater than or equal to the inter-electrode distance between the electrodes from any single pair.

The electrodes can be implanted subserosally as illustrated in FIGS. 7(b-c) or from the mucosal side as illustrated in FIG. 7(a). In the illustrated electrode configurations, electrodes implanted on the posterior wall of the organ are lighter in color for purposes of clarity of presentation. In some embodiments, the electrodes of a given set are arranged correspondingly to imaginary lines perpendicular to the organ axis (which is also shown in lighter color).

The number and placement of electrodes is dependent upon the treatment protocol. For example, in some embodiments, the delivered electrical stimulation overrides the spontaneously existing slow waves and produces artificial retrograde peristalsis. The electrical stimulation achieves artificial retrograde peristalsis independently from the underlying myoelectrical activity and the spontaneously existing electromechanical phenomena at the stimulated site.

In this embodiment one or more pairs of electrodes are implanted on or in the vicinity of the distal sphincter of an organ of the GI tract, such as, for example, in the pre-pyloric area of the stomach as illustrated in FIG. 7. In one embodiment a single stimulation channel is utilized to deliver NGES pulses to the set of electrodes implanted in the pre-pyloric area of the stomach. In this embodiment the delivered pulses electrically stimulate cholinergic nerves in the area of the implanted electrodes which triggers the release of acetylcholine (ACh) in the vicinity of the implanted electrodes.

The released ACh induces a local lumen occluding contraction of the circumferential area of the organ encompassing the implanted electrodes which, in some embodiments, is propagated in the retrograde direction for 6-10 cm. The invocation of retrograde contractions or reverse peristalsis provides at least partial control over GI organ emptying (for example, gastric emptying) and thus, indirectly, satiety and food intake.

In this embodiment, the implanted stimulation device delivers NGES pulses having an amplitude in the range of 3-30 volts (peak-to-peak) and at a frequency in the range of between 5 to 50,000 Hz (period of the rectangular pulses from 20 us to 0.2 s). The duty cycle of the delivered stimulation pulses can vary between 1% and 100%. The duty cycle is expressed in percent as 100 times the ratio of the pulse duration P to the period of the signal. In one embodiment the stimulation pulses are bipolar pulses having a rectangular waveform. In the case of a bipolar signal, the pulse duration P includes both the positive and negative portions of the pulse. However, unipolar or multi-polar pulses having any of a variety of waveforms commonly known in the art may also be used.

The duration (i.e. on time) of each batch or train of stimulation pulses ranges from between about two seconds to eight seconds. The time between delivered trains of pulses can vary between two seconds and twenty four hours, followed by another train of stimulation pulses.

However, smooth muscle has a cyclic response to prolonged neural electrical stimulation. In operation, prolonged continuous stimulation exhaust the ACh deposits in the vicinity of the implanted electrodes resulting in relaxation of the smooth muscle and loss of contraction regardless of applied stimulation.

Nevertheless, when properly timed and parameterized, NGES pulses applied to the pre-pyloric electrode in accordance with one embodiment of the present invention repetitively induces a lumen occluding pre-pyloric contraction, which propagates in a retrograde fashion. The scope, frequency and the magnitude of the induced retrograde contractions depend on the magnitude, frequency and duty cycle of the neural stimulation pulses. In addition, the duration of the pulse train (i.e. the on-time) also affects the strength and scope of the retrograde contractions.

For example, in one illustrative embodiment an implantable stimulation device is coupled to a single pre-pyloric set of electrodes in the pre-pyloric area of a patient's stomach as illustrated in FIGS. 7(b-c). In this embodiment the implanted device is programmed to deliver a six second train or burst of bipolar rectangular pulses having a 16 volt (peak-to-peak) amplitude, 60% duty cycle at a frequency of 50 Hz.

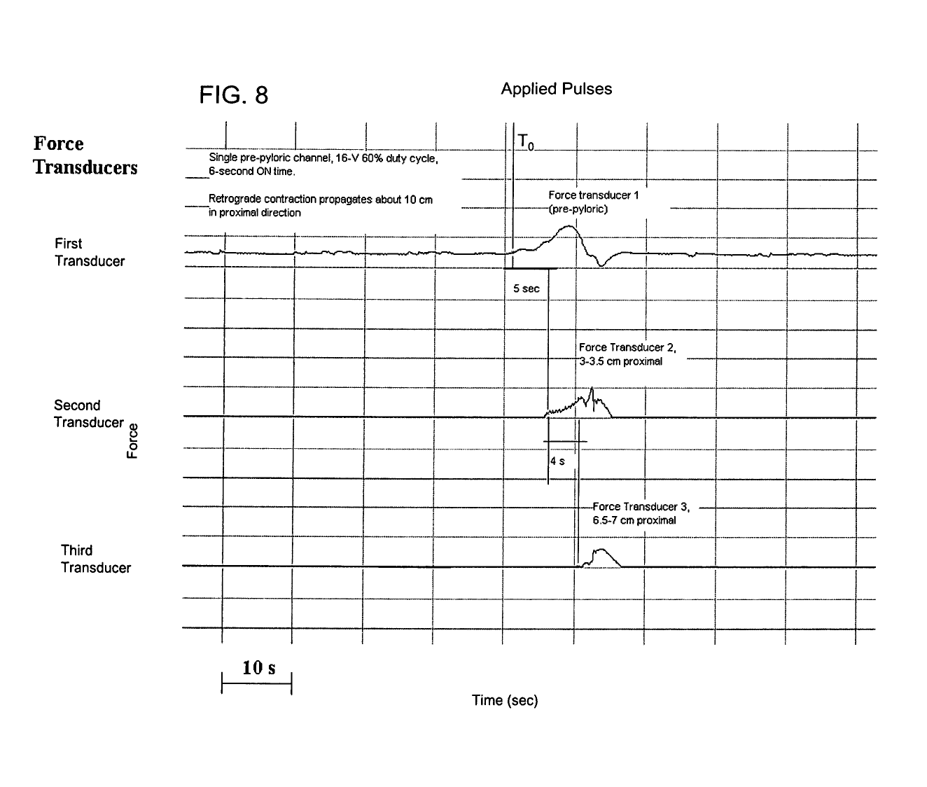

FIG. 8 graphically illustrates the force measured by three transducers located proximal to the implanted electrodes as a function of time. As illustrated in FIG. 8, the stimulation pulses, delivered at time T₀ induce a contraction in the circumferential vicinity of the implanted electrodes. The induced contraction then propagates in a retrograde direction as illustrated by the contraction force measured by a second transducer located 3-3.5 cm proximally to the implanted electrodes and a third force transducer located 6.5-7 cm proximally to the second force transducer.

The magnitude of the retrograde contractions (in relative units), in accordance with this embodiment, typically exceeds the magnitude of spontaneously recorded contractile activity at the location of the stimulating electrode set, indicating that stimulation with lower amplitude and/or duty cycle can also be used to invoke contractions of physiological strength or lower. Indeed, in other embodiments stimulation pulses with various voltage amplitudes, various durations, and various duty cycles can be used to produce different contractile responses.

In some embodiments, therefore, one or more force transducers are implanted in a proximal direction (i.e. above the electrodes toward the fundus) to the pre-pyloric electrode set to measure the strength of the induced retrograde contractions. In these embodiments the microcontroller monitors the force of the contractions induced at one or more locations and adjusts the stimulation parameters to control the scope and strength of the retrograde contractions.

In one embodiment the off time, i.e. the time during which no stimulation is applied to the smooth muscle in the pre-pyloric area, is on the order of approximately five minutes. At the end of a five minute off time the smooth stomach muscle is sufficiently recovered to allow for the induction of a subsequent contraction which is also propagated in the retrograde direction in response to a subsequent pulse train.

One of skill in the art will appreciate that the present invention is not limited to a particular stimulation protocol. Rather, the stimulation parameters, including amplitude, frequency, duty cycle, as well as the length of a stimulation pulse train and off time between pulse trains may be varied to produce contractions of varying force and scope as may be desired for a particular treatment protocol.

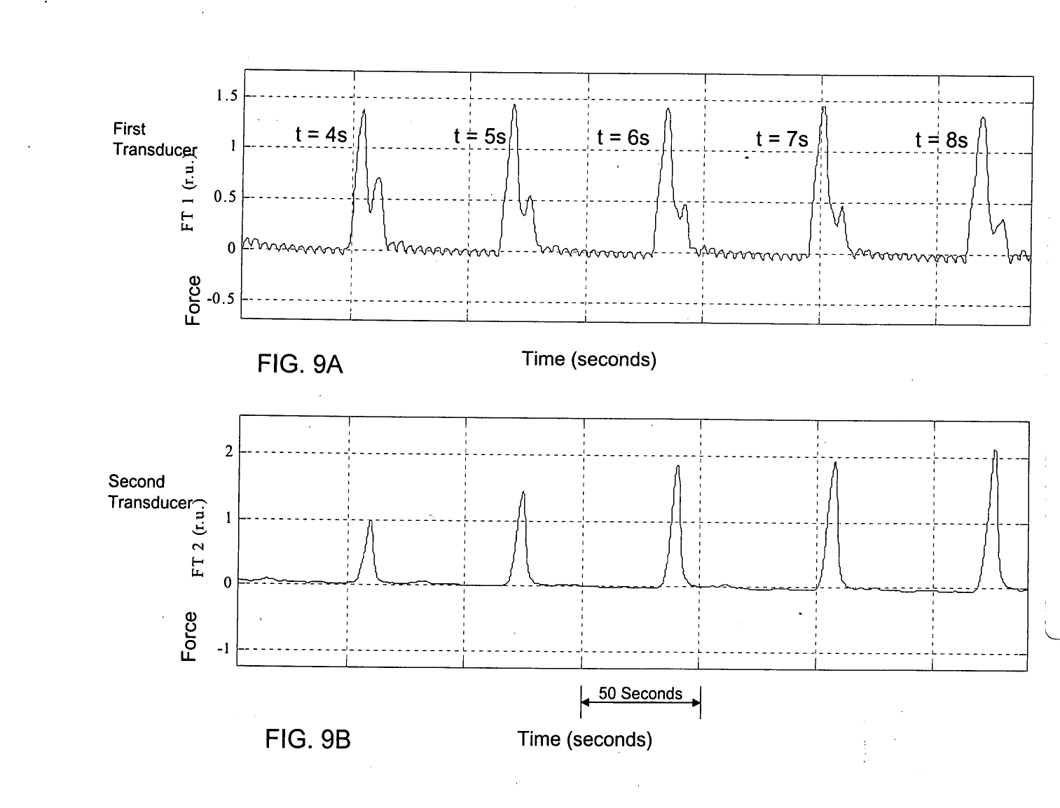

For example FIG. 9 graphically illustrates the interaction between the duration of the stimulation pulse train and the strength of induced retrograde contractions at two locations proximal to a single pre-pyloric electrode set. In this illustrative example the rectangular pulse trains having a duration varying from 4-8 seconds having a 16 volt (peak-to-peak) amplitude, 100 % duty cycle at a frequency of 50 Hz. As illustrates, longer stimulation durations induce more powerful local gastric contractions and better retrograde propagation of these contractions. However, there is a finite limit for the contractile response of the tissue to longer durations of stimulation. Under sustained electrical stimulation, the tissue stops contracting after some time and enters a refractory period.

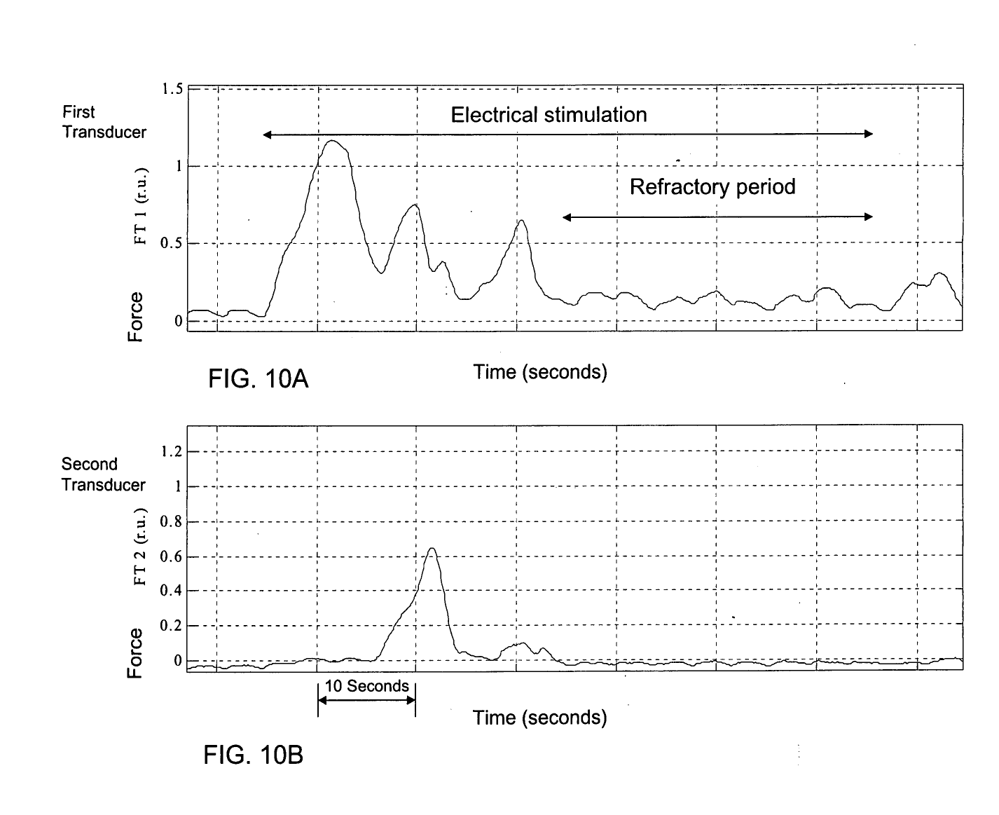

For example, FIG. 10 graphically illustrates the force as a function of time at two locations proximal to a single pre-pyloric electrode set under sustained electrical stimulation. In this example, the stimulated tissues enters a refractory period after approximately 30 seconds of continuous stimulation with pulses having a 16 volt (peak-to-peak) amplitude, 100% duty cycle at a frequency of 50 Hz.

In addition, in some embodiments of the present invention the implantable device delivers neural gastric electrical stimulation to produce artificial contractions which disrupt the spontaneously-existing antegrade peristalsis to induce temporary paresis of at least a portion of the GI tract. The temporary paresis delays gastrointestinal transit of food content in a controlled fashion, in such a way that the patient or subject experiences lack of appetite, early satiety, and unwillingness to consume more than a pre-determined amount of food.

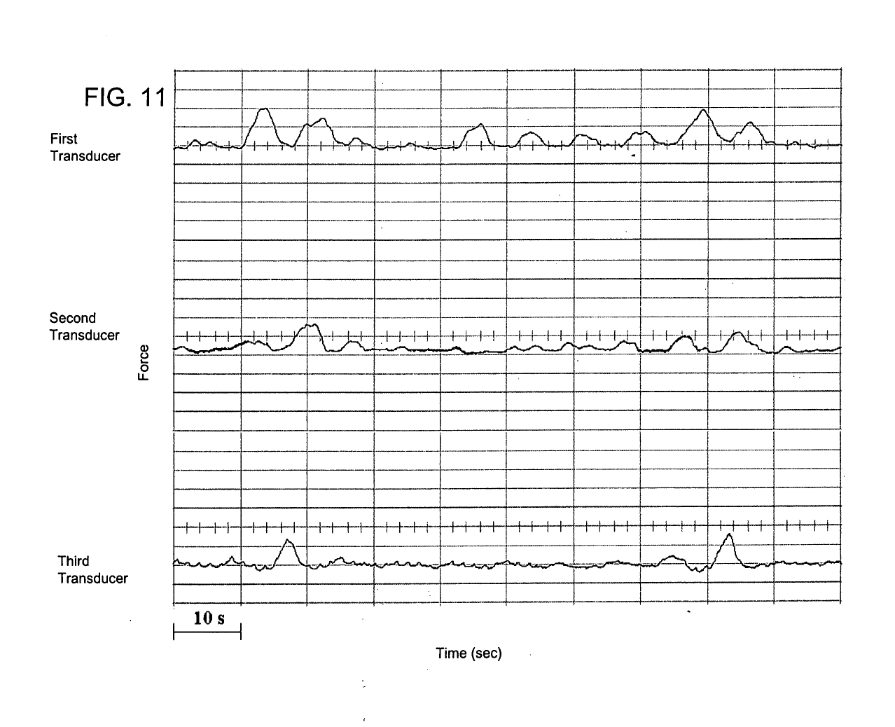

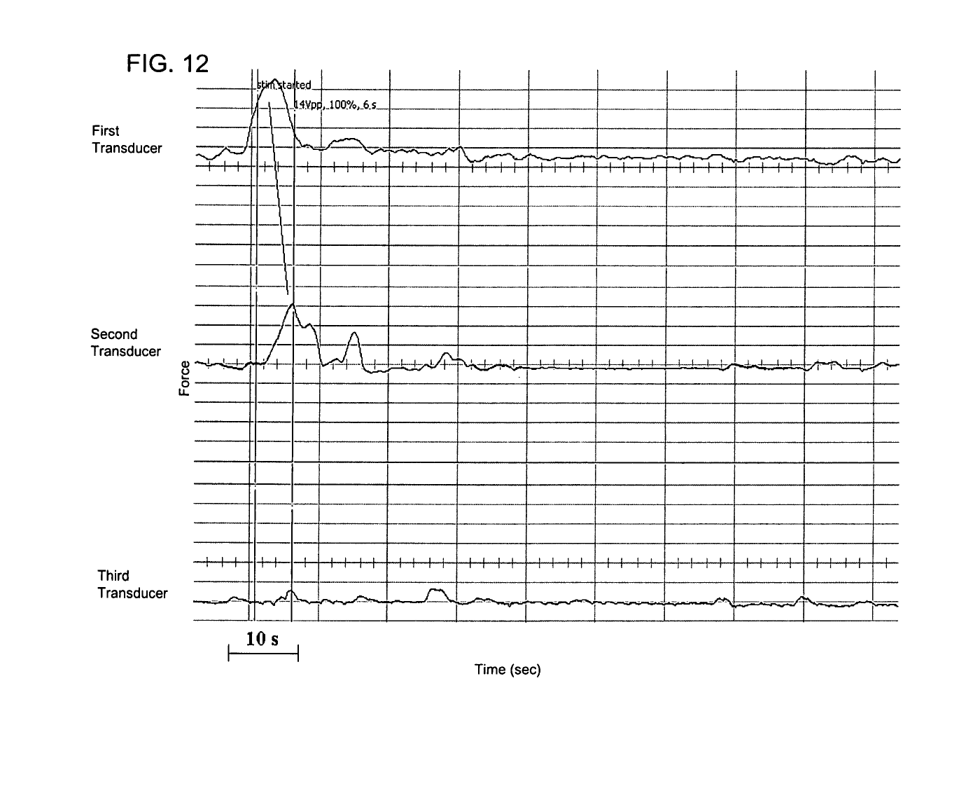

For example, FIG. 11 graphically illustrates strong content-propulsive intrinsic contractions typically seen in the GI tract at three locations proximal to the pylorus. In one embodiment, an implantable pulse generator is again coupled to a single pre-pyloric set of electrodes in the pre-pyloric area of a patient's stomach. In this embodiment the implanted device is programmed to deliver a six second train or burst of bipolar rectangular pulses with a 14 volt (peak-to-peak) amplitude, 100% duty cycle at a frequency of 50 Hz to induce temporary paresis as demonstrated in the force measurements at three proximal location illustrated in FIG. 12.

One of skill in the art will further appreciate that the present invention is not limited to a particular implantation site or organ. Rather the present invention may be utilized to induce contractions in any smooth muscle organ. However, the number of electrode pairs will typically vary in accordance with the circumference of the organ in the area where the electrodes are implanted.

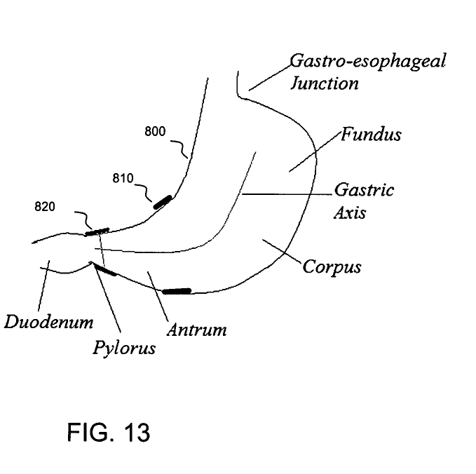

For example, the present invention may be implemented utilizing one or more sets of electrodes 810 implanted in any area of the stomach that is proximal to or above the pyloric area as illustrated in FIG. 13. In this embodiment, the number of electrode sets and the level of stimulation energy applied is, by way of example, adjusted to again induce a contraction in the circumferential area around the implanted electrodes as previously described. However, for electrodes implanted in the upper circumferential area of the stomach the induced contraction propagates in the antegrade direction creating a propulsive force in the direction of the pylorus. Artificially induced antegrade contractions may therefore be useful as a treatment for gastroparesis, constipation, or other motility disorders.

Alternatively, the implantable pulse generator may adjust the stimulation parameters to control the propulsive force of the induced antegrade contraction to minimize its impact on gastric emptying. In this embodiment, the applied stimulation may also be adjusted to induce artificial paresis in at least a portion of the stomach to again control the transit of food content through the stomach and to induce an early sensation of satiety.

In another embodiment the implantable pulse generator includes a second stimulation channel coupled to a second set of electrodes 820 implanted in the pyloric sphincter area. In this embodiment the implantable pulse generator applies electrical stimulation to the second set of electrodes to control the contraction force of the sphincter to further control gastric emptying.

For example, in one embodiment the implantable pulse generator applies stimulation pulses to the second set of stimulation electrodes 820 prior to or otherwise in cooperation with the stimulation applied to the first set of electrodes 810 to increase the force of contraction of the sphincter to mitigate the propulsive effects of induced contractions which artificially propagate in the antegrade direction away from the first set of electrodes 810.

Alternatively, the implantable pulse generator may apply stimulation pulses to the second set of electrodes 820 to exhaust the smooth muscle in the area of the implanted electrodes and to reduce the force of the sphincter contraction. In this embodiment the implantable pulse generator then applies stimulation pulses to the first set of electrodes to induce a contraction that artificially propagates in the antegrade direction, propelling food content out of the relaxed sphincter as a treatment for gastroparesis or other motility disorders.

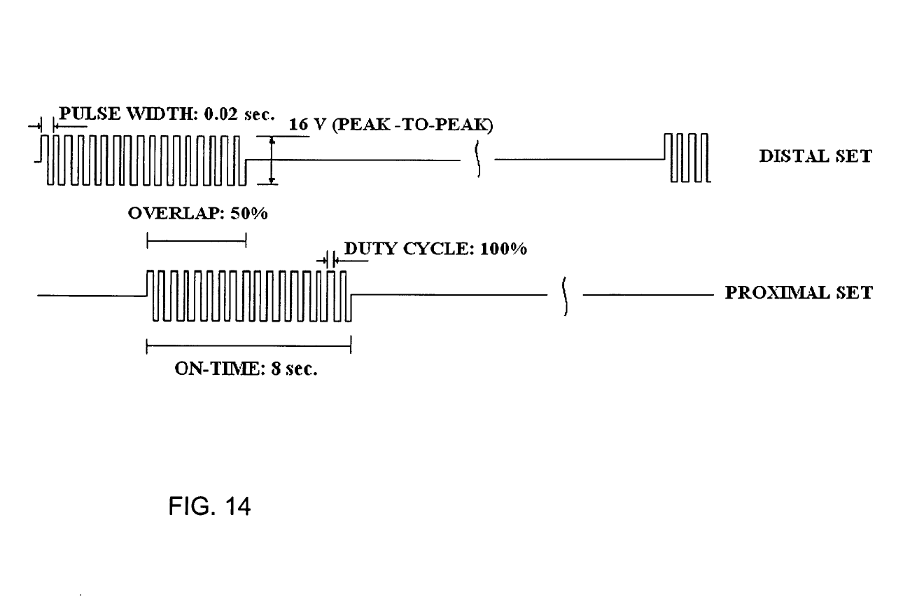

Alternatively, in one embodiment an implantable pulse generator is adapted to deliver electrical stimulation in a predetermined sequence to the two different electrode sets to increase the propagation distance of an artificially induced contraction. For example, in one embodiment a first electrode set is implanted in the pre-pyloric area and a second set is implanted in the gastric antrum located about 4 cm proximal to the first electrode set (see FIG. 13). In this embodiment the implantable pulse generator delivers overlapping stimuli pulse trains to the first and second electrode sets. In one embodiment the overlap of the stimulation pulses is approximately 50% of the on time of the pulse train as illustrated in the stimulation pattern of FIG. 14. One of skill in the art will appreciate however that the stimulation overlap may also be varied to control the strength and propulsive force of the induced contraction.

Similarly, in another embodiment the implantable pulse generator monitors the underlying intrinsic myoelectric activity of the organ under stimulation and applies electrical stimulation to the organ through one or more electrode sets in synchrony with the underlying slow wave. In this embodiment, the electrical stimulation may be used to increase the force of a contraction or increase the duration of an intrinsic contraction. Hereto, the delivered stimulation energy, as controlled by the pulse amplitude, frequency, duty cycle and on and off times of the pulse trains can be adjusted to induce paresis in the stimulated organ for a predetermined period of time.

While the invention herein disclosed has been described by means of specific embodiments and applications thereof, numerous modifications and variations could be made thereto by those skilled in the art without departing from the scope of the invention set forth in the claims. For example, the methods or algorithms described in connection with the embodiments disclosed herein may be embodied directly in hardware, in a software module executed by a processor, or in a combination of the two.

A software module may reside in RAM memory, flash memory, ROM memory, EPROM memory, EEPROM memory, registers, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention.

What is claimed is:

1. A method for controlling motility in a gastrointestinal tract, comprising:
applying electrical stimulation to a set of electrodes implanted in a predetermined region of the gastrointestinal tract to induce a circumferential contraction that propagates in a predetermined direction.

2. The method of claim 1 wherein applying electrical stimulation to a set of electrodes comprises applying neural gastric electrical stimulation to a set of electrodes.

3. The method of claim 1 wherein the electrodes are implanted in a pre-sphincter region of the gastrointestinal organ.

4. The method of claim 3 wherein the induced contraction propagates in a retrograde direction.

5. The method of claim 1 further comprising the step of determining whether normal motility has been restored in the predetermined portion of the gastrointestinal tract.

6. The method of claim 5 further comprising the step of using the results of the determination of claim 5 to decide whether to apply further electrical stimulation to the set of electrodes.

7. A method for controlling motility in a gastrointestinal tract, comprising:
applying electrical stimulation having a first set of stimulation parameters to a set of electrodes implanted in a predetermined region of the gastrointestinal tract to induce paresis in at least a portion of the gastrointestinal tract for a first period of time.

8. The method of claim 7 further comprising measuring a physiologic response of the gastrointestinal tract to the electrical stimulation having a first set of stimulation parameters and applying electrical stimulation having a second set of stimulation parameters to a set of electrodes implanted in a predetermined region of the gastrointestinal tract to induce paresis in at least a portion of the gastrointestinal tract for a second period of time.

9. The method of claim 8 wherein the stimulation parameters include at least one of pulse amplitude, duty cycle, on time of pulse trains and off time between pulse trains.

10. The method of claim 8 wherein a predetermined algorithm determines the second set of stimulation parameters.

11. An apparatus for controlling gastrointestinal motility, comprising:
a set of electrodes suitable for implantation in a predetermined region of a gastrointestinal tract; and
a controller adapted to deliver electrical stimulation to the set of electrodes to induce a circumferential contraction that propagates in a predetermined direction.

12. The apparatus of claim 11 wherein the controller is adapted to deliver a neural gastric electrical stimulation to the set of electrodes.

13. The apparatus of claim 12 wherein the set of electrodes are implanted in a pre-sphincter region of the gastrointestinal tract.

14. The apparatus of claim 11 wherein the controller is adapted to propagate the circumferential contraction in a retrograde direction.

15. The apparatus of claim 11 further comprising one or more sensors for measuring a physiological response of the gastrointestinal tract to the delivered electrical stimulation.

16. The apparatus of claim 15 wherein the controller is adapted to determine, from signals from the one or more sensors, whether intrinsic motility has been restored in the predetermined portion of the gastrointestinal tract.

17. The apparatus of claim 16 wherein the controller is further adapted to apply further electrical stimulation to the set of electrodes in response to detection of intrinsic motility.

18. The apparatus of claim 15 wherein the one or more sensors comprise at least one of sensing electrodes or mechanical sensors.

19. The apparatus of claim 18 wherein the mechanical sensors are selected from a group consisting of force transducers, strain gauges and pressure gauges.

20. The apparatus of claim 15 wherein the controller is adapted to adjust a set of electrical stimulation parameters in response to an output of the one or more sensors.
